(19) United States
(12) Patent Application Publication
(10) Pub. No.: US 2020/0364182 A1
(43) Pub. Date: Nov. 19, 2020

(54) METHOD AND SYSTEM FOR OBSERVING, RECORDING, STORING, MONITORING, TRACKING, CORRELATING, AND ANALYZING HUMAN PHENOTYPE DATA FOR THE PURPOSES OF MEDICAL INTERVENTION OPTIMIZATION, NON-MEDICAL INTERVENTION OPTIMIZATION, AND IMPROVED HUMAN GENOTYPE DATA UTILITY.

(71) Applicant: William Taylor Threlkeld, West Palm Beach, FL (US)

(72) Inventor: William Taylor Threlkeld, West Palm Beach, FL (US)

(21) Appl. No.: 16/410,717

(22) Filed: May 13, 2019

(51) Int. Cl. G06F 16/13; G16H 80/00

(52) U.S. Cl. CPC G06F 16/13; G16H 80/00

(57) ABSTRACT

This invention is a method and system for comprehensive observing, recording, storing, monitoring, tracking, correlating, and analyzing human phenotype data. All human phenotype information is centrally stored and remotely accessible. This method allows the optimization of medical and non-medical intervention activity by providing a means for data analysis of intervention efficacy reducing financial burden to the patient and their caregivers. Human phenotype data is aggregated and updateable allowing for research participation and highly informative tracking.

### CROSS-REFERENCE TO RELATED APPLICATIONS

Provisional Patent Application No. 62/670,830

### FEDERALLY SPONSORED RESEARCH

None

### SEQUENCE LISTING

None

### BACKGROUND

### Field

The invention relates to a method and system for improved human phenotyping, specifically for compressing the timeline for human medical research discovery, improving healthcare intervention efficacy, economy, and management.

### Prior Art

“A phenotype is the composite of an organism's observable characteristics or traits, such as its morphology, development, biochemical or physiological properties, behavior, and products of behavior” (Wikipedia). A phenotype in humans can be as common as eye color or as complex as an individuals' genotype interaction with the environment causing disease. Generally, if a human is healthy, the consumption of genotype and phenotype data is recreational. Where human disease is present, phenotyping is used, and a systematic method commences; symptoms, observations, assessment and treatment plan (SOAP). More specifically, the observation and recording of the symptoms is medical phenotyping. This is accomplished through observations of the individual by medical expert(s) (human beings), and often includes qualified diagnostic test(s) (non-human) where the results are interpreted by a medical doctor and/or specialist. The assessment is the selection of an appropriate intervention (if known) based on the assumed phenotype. The treatment plan is the implementation of the selected intervention(s) to mitigate and/or resolve the disease. The SOAP process is the logical basis of human healthcare education, training, care, and treatment.

Healthcare treatment and medical discovery through research has several systemic issues; fragmented medical records and research data, insufficient observation (phenotype) time with healthcare experts, rising cost of healthcare interventions, inadequate phenotype research data collection and sharing methodology to insure justice for research participants, and advances in genetic testing technology outpacing phenotype collection methods making genotype data more affordable for patients/clients, but not more useful due to poor methods to collect, store, and verify the corresponding phenotype data efficiently.

One of the novel Affordable Health Care Act (ACA) requirements is that medical records be digitized and made accessible to patients. One of the complications that remains in implementation of this provision of the ACA is continued fragmentation of the health records. This is due to healthcare providers using a variety of IT contractors, storage connectivity standards, and the lack of integration for those providers that choose not to participate (opting to pay the fine). The current digital medical record system remains inefficient because it has yet to address this deficiency uniformly. Provider to provider inter portal integration, non-participating provider data, and legacy data access remain elusive to patients and caregivers. The integration of healthcare records has been discussed by various papers and tech companies (Apple) however the proposed solutions are passive in nature “opt in with participating network providers” and is incomplete. Continuous human phenotyping requires this utility. Having complete patient records in a centralized accessible location cannot be viewed as merely a convenience to the patient where the treatment and study of human disease is concerned, but a requirement for reduction of error in phenotype diagnostics, redundancy, intervention efficacy assessment and the identification of potential influence(s) of environmental epigenetic factors on the intervention(s) being applied.

Insufficient time will medical experts remains a systemic issue with healthcare. The risk for errors in assessment of intervention efficacy are reduced where the treatment plan and intervention latency is known through well-established phenotype and intervention data sets. For example, if the phenotype is arm impairment observed to be a fractured skeletal structure and confirmed by x-ray diagnostic, the treatment plan (intervention) is for the affected bone to be set, the arm to be immobilized and revisited once healing has concluded. The fractured arm treatment plan is established, and intervention efficacy is known.

In a case involving the application of mood-altering intervention activity (antidepressant prescriptions), intervention latency is established through the datasets that exist to support that the intervention may take several weeks to reach optimum effectiveness to treat the disorder. In more complex disease where causal and treatment plans lack sufficient data, such as autism spectrum disorders and most forms of dementia, intervention application efficiency, efficacy, and potential latency are not known. Further compromising the treatment plan options with more complex disease is the time a patient spends with medical expert(s) for phenotyping. Under most circumstances, as little as a few minutes a visit under observation, is not sufficient to capture the genotypes' interaction with the pathology of disease let alone consider the epigenetic impact on the current or proposed intervention(s) efficacy.

Although the therapeutic outcome for any given patient cannot yet be made certain, errors in initial and ongoing assessment derived from insufficient and poor phenotype data contribute negatively to outcomes in medical research discovery for potential treatment(s)for disease. The more complex and chronic the disease, the greater need for ongoing phenotype observation data and analysis.

The rising cost of each health care intervention is well documented, and the causes are numerous. In complex disease or where multiple interventions are being applied simultaneously, optimization of a treatment plan accomplished by improved phenotype monitoring creates economy. For autism, many interventions are prescribed for the patient simultaneously by the medical expert community; speech therapy, occupational therapy, diet modification, and pharmaceutical. The non-medical/holistic community also markets potential solutions. Even with insurance and government funded subsidies, the patient and/or their caregivers is left with a substantial financial burden in medically prescribed interventions and the full financial burden of all non-medical/holistic interventions. In most cases, the caregiver attempts to assess intervention efficacy and efficiently coordinate their timing is attempted via note taking (paper notes and/or non-integrated digital notes). While all caregiver effort is admirable, this method becomes cumbersome, relevant analysis cannot be made in real time, makes doctors' visits less productive, and ultimately is not feasible long-term. To create intervention optimization and economy, the correlation of therapies and their interrelated efficacy needs to be established through the analysis of combined data sets in real time.

One of the provisions of the “Belmont Report” is justice “burdens and benefits” for the participants in human research. Justice states: “1) to each person an equal share, 2) to each person according to individual need, 3) to each person according to individual effort, . . . ” In current research study methods, the participants are often the last to know the findings of the research or benefit directly. It is important to understand why a patient would willingly serve as a research subject. The patient is a willing participate because of one or more of the following: monetary compensation, inherent dissatisfaction with the medical treatment options available, and altruism. The research study findings (often delayed), are typically depicted through academic and/or other published works but not always. The value of altruism on the part of research participants cannot be overstated. However, the lack of direct and timely benefit for research subject participation does not increase optimism, hope (for all stakeholders), and valid subject participation.

Facilitating research participants, their caregivers, and other stakeholders with continuous real time feedback on current patient intervention efficacy and progress is advantageous because credible data may be captured and made available that justifies modifications to the treatment plan in turn creating maximum patient and social benefit. The benefit is compounded when the individual patient data is correlated with the de-identified intervention efficacy data of the other study group participants, and at different ages and stages of disease. The ethicality of medical research is further validated when data is shared that benefits study participants according to individual effort and need.

Advances in human genetic mapping and testing technology have created substantial data analysis issues. The abundance of genotype data has limited utility until it is correlated with the relevant phenotype data. The feasibility of obtaining and combining phenotype data from a variety of sources has constraints that need remediation; varied observer expertise, observer bias, and the burden to the observer of recording and managing their observations. To remediate the bias and expertise, the phenotype data must be normalized across observers by a relevant data input weighting accomplished it such a way that does not discourage participation due to resource burden. To reduce the time burden on the observer and make observation entry more consistent, the observation workload must be separated into manageable tasks. Finally, the phenotype system needs a utility that facilitates accuracy of the phenotype data by improving the accuracy of participant observers regardless of the observer's level of expertise.

Insofar as we are aware, no method and system for comprehensive observing, recording, storing, monitoring, tracking, correlating, and analyzing human phenotype data for the purposes of improved human phenotyping, specifically for compressing the timeline for human medical research discovery, improving medical intervention optimization, non-medical intervention optimization, increasing human genotype discovery value, and healthcare economy and management exists.

### SUMMARY

In response to the need for continuous human phenotyping, the current invention the first ever patient and research subject oriented system and method for observing, recording, storing, monitoring, tracking, correlating, and analyzing human phenotype data. The applications and advantages of the current invention can be understood from a study of the following description and accompanying drawings.

### BRIEF DESCRIPTION OF THE DRAWINGS—FIGURES

FIG. 1 is a process/workflow diagram of medical record creation and stored in various fragmented databases.

FIG. 2 is a process/workflow diagram of a medical record data consolidation utility allowing the patient a singular portal to view all their medical records.

FIG. 3 is a process/workflow diagram of a human phenotype observation data entry, records entry, and storage system being accessed remotely, updated, merged and appended.

FIG. 4 is a diagram of user roles and phenotype observation participation.

FIG. 5 is a diagram of scheduled phenotype observation by category.

FIG. 6 is a process/workflow diagram of a human phenotype data output for reporting submission review, aggregated and correlated data for single subject analysis, group subject analysis, being accessed remotely, updated, merged and appended.

FIG. 7 is a process/workflow diagram for compensating for human observer bias in subjective human phenotype observations.

FIG. 8 is a process/method for human phenotype observation data normalization uses a scoring system.

FIG. 8A is a screenshot example of how a uniform scoring system would be depicted, accessed remotely, updated, merged, and appended.

FIG. 9 is a screenshot example of a human phenotype observation submission frequency monitoring output, accessed remotely, updated, merged, and appended.

FIG. 10 is a process/workflow diagram of a neural network training system for human phenotype prediction accessed remotely, updated, merged, appended, and backpropagated.

FIG. 11 is a process/workflow diagram of a neural network training system for human phenotype prediction to improve the accuracy of both the human phenotype observer and the neural network, accessed remotely, updated, merged, appended, and backpropagated.

### DETAILED DESCRIPTION

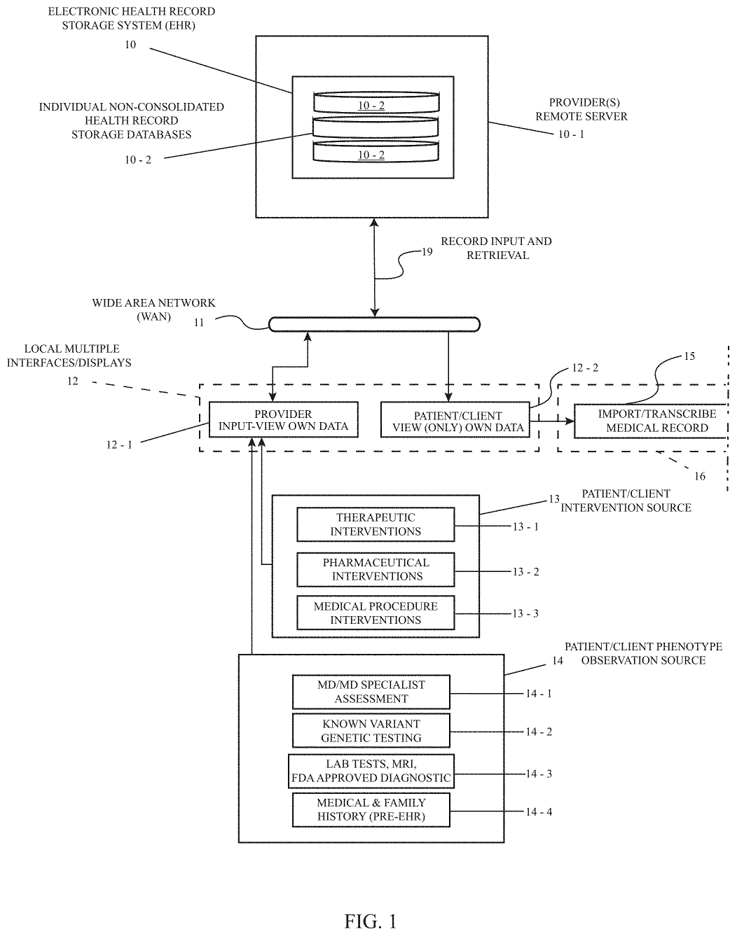

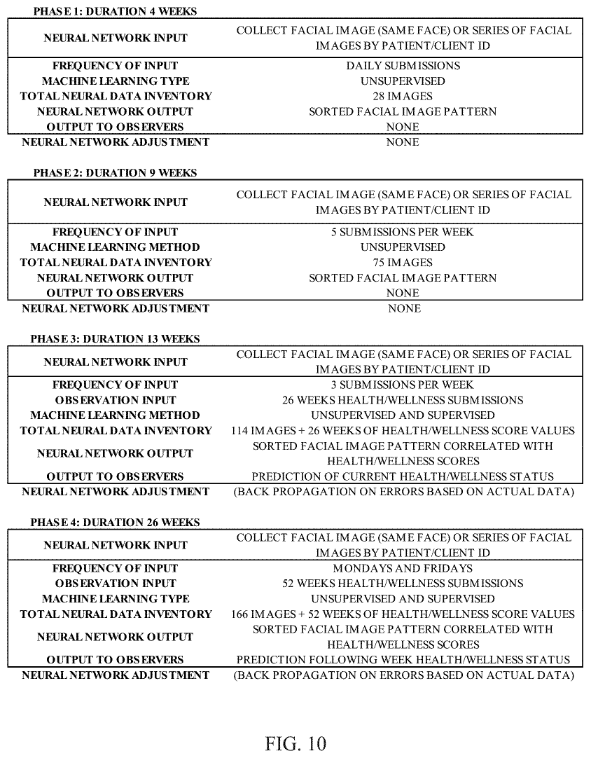

FIG. 1 contains the depiction of a digital health record management system and one embodiment of our method 15 of transferring health record phenotype and intervention data to a singular database. In FIG. 1 health care providers document patient health history 14-4, perform and document various medical diagnostic information 14-2/14-3, and document applicable clinical findings through human observation. The resulting phenotypic data is then entered into provider portal 12-1 through local multiple displays 12 and transmitted by way of WAN 11 to a provider specific health record storage system 10 consisting of a database 10-2 and remote server 10-1. As a patient undergoes interventions 13, the therapeutic intervention 13-1, pharmaceutical intervention 13-2, and medical procedures 13-1 are document and transmitted to 10 on occurrence then entered into provider portal 12-1 through local multiple displays 12 and transmitted by way of WAN 11 to a provider specific health record storage system 10 consisting of a database 10-2 and remote server 10-1. The provider can retrieve its own entered data using 12-1 via 12. The patient can view his/her own medical record(s) via various patient portals 12-2 made accessible by various medical providers. An import and transcribe interface 15 is depicted as an initial step of consolidating patient data FIG. 2.

FIG. 2 contains one embodiment or electronic medical record consolidation. In FIG. 2 non-consolidated medical record data is imported/transcribed through utility 15 accessed via 16 by way of application programming interface or manual transcription. The non-consolidated medical record data 17 is transmitted through WAN 11 to remote server 18 where the digitized health record storage 21 is processed 20-1 to consolidate all patient health data by unique patient identification. Inputting and retrieving data 19 is then accessible through client/patient consolidated view 20.

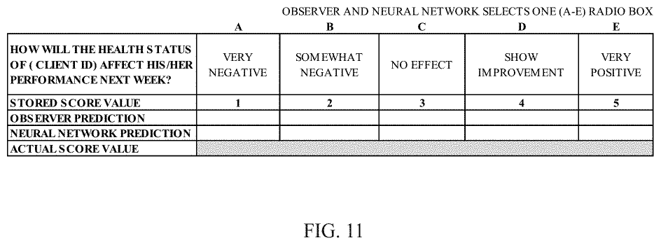

FIG. 3 contains one embodiment or method and system inputs 24 for human and non-human phenotype observation data recording, storage, monitoring, tracking, analyzing and correlating efficacy of medical and non-medical interventions. In FIG. 3 system input 24 is comprised of non-system data 17, objective human and non-human phenotype data 25 including 26-36 entries/submission types, subjective phenotype human observer submission 38, comprising 39-43 data submission types outside research database(s) 44 accessible via provider application program interface (API) 45 held on providers remote server 18, and machine learning inputs 37. The data is entered into the system through multiple local interfaces/displays 16 filtered entry 46 by user role and patient client ID. Once data is entered it travels on a secure pathway 23 via WAN 11 to remote a remote server 18 that house the patient/client data storage and phenotype applications 21 consolidated individual health record storage database 21 subdivided into 21-A-21-F.

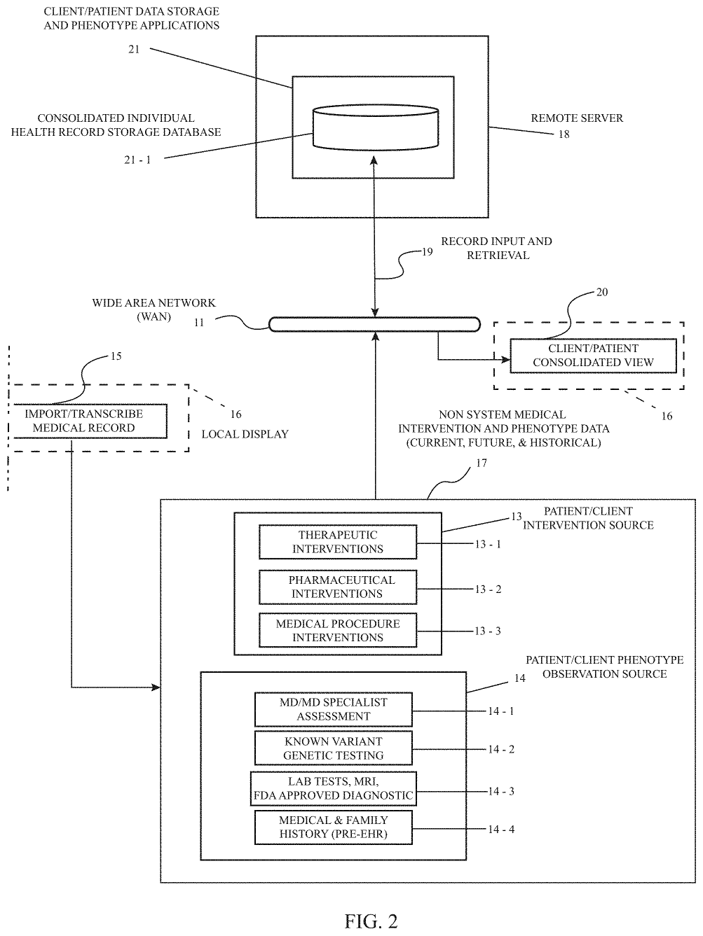

FIG. 4 contains one example of human phenotype observers and their participation observation categories 410.

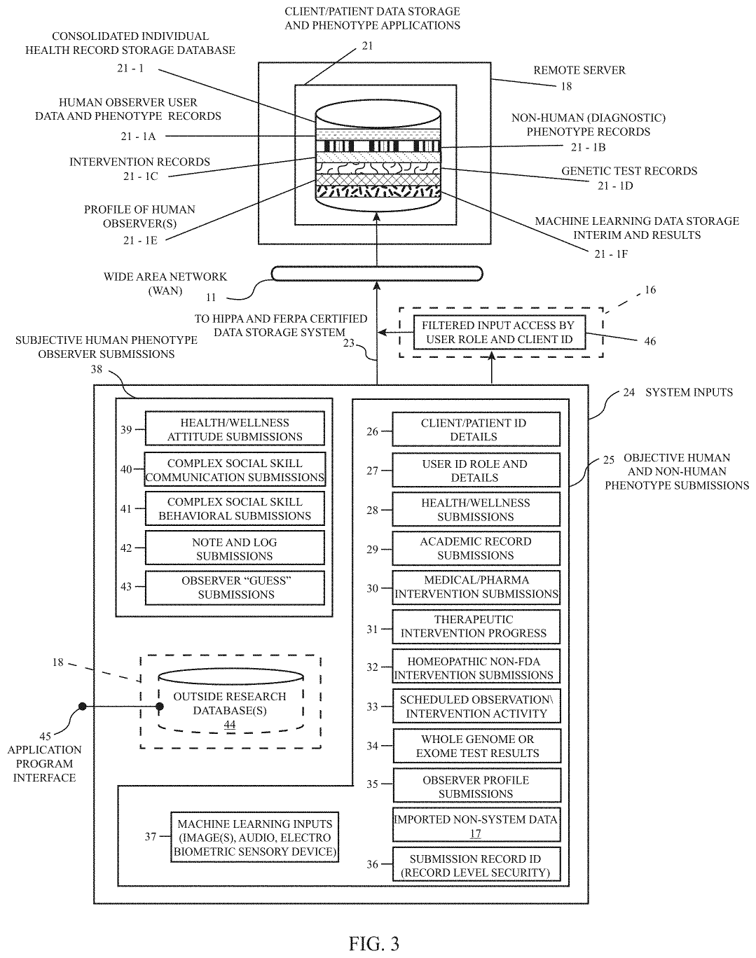

FIG. 5 depicts an example of observation frequencies and datapoints for various phenotype data gathering.

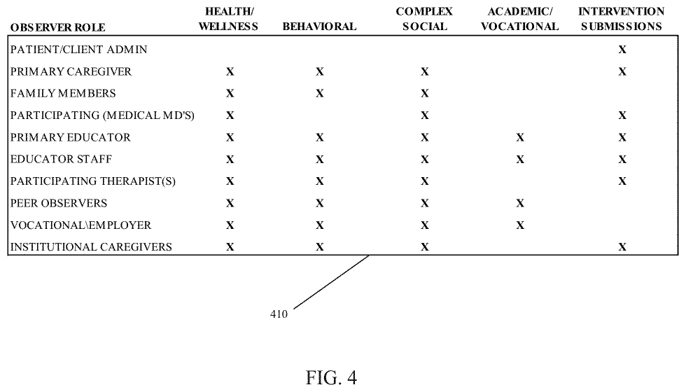

FIG. 6 contains one embodiment or method and system for retrieval/outputs 47 of human and non-human phenotype observation data recording, storage, monitoring, tracking, analyzing and correlating efficacy of medical and non-medical interventions. In FIG. 6 stored data 21-1 and subparts 21-1A-21-1F travel from remote server 18 through WAN 11 to system output 47 that is comprised of submission record detail 48 and subparts identified sequentially as 49-60. Individual and clustered data 61 consisting of subparts 62-70 are accessed for viewing or printing through interface 46 viewed locally local interface 16. Linear and non-linear correlations are performed on individual data to determine intervention efficacy and reports 67 are created. Intervention efficacy individually is then compared to correlated and aggregated data, deidentified, and filtered by phenotype 69. Human observer training data is accomplished by feeding back the machine learned response and the human guess response for individual evaluation and insight 68.

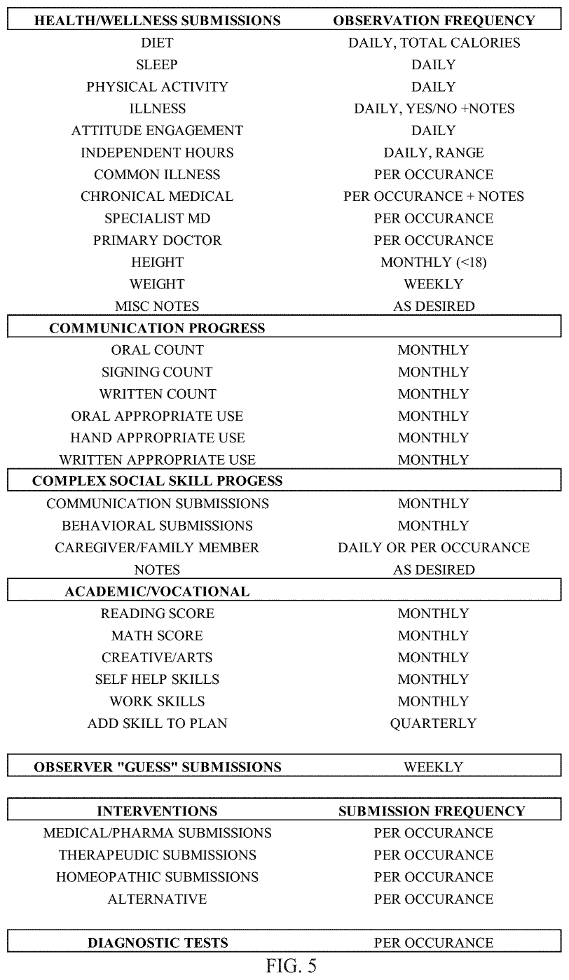

FIG. 7 contains one embodiment or method and system for human phenotype observer evaluation and training to improve reliability and improve the accuracy of human observations. In FIG. 7 new user data records 722 are collected. Each new user record has a date stamp 718, the user is assigned a unique user ID 719, and user contact details 720, and human phenotype observer role 721. The initial human observer data 710-A is comprised of education level 714, professional experience 715, non-professional experience 716 and additional observer traits 717 that may be relevant to the human phenotype study and is used as a starting point value for the observer expert score 710. The new user records are transmitted over WAN 11 to a remote server 18 and store in 21-1 and subparts 21-1A, 21-1B. The current user expert score is calculated in application 21, and transmitted to current expert score 710. Modifications either positive or negative are influenced by data 710-B comprised of submission punctuality 711, observation human guess compared to actual result and machine learned prediction 713, and actual therapeutic patient client progress 713. 710-B data is applied and the current observer expert score 710 is adjusted.

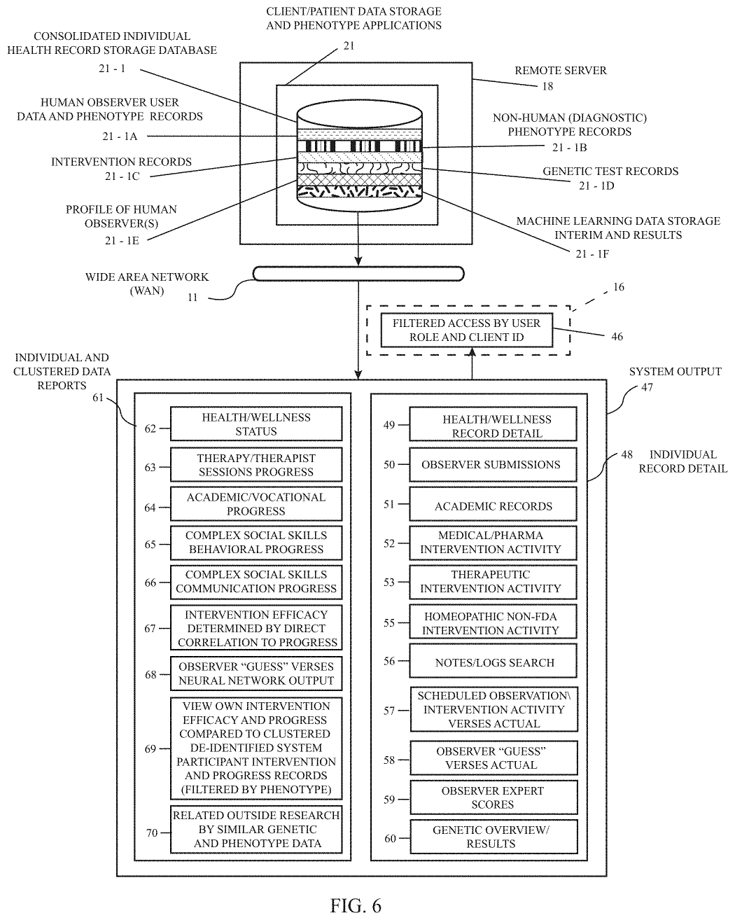

FIG. 8 is an example of a scoring system assigning numerical values to phrases so that normalization can be achieved through various math calculations.

FIG. 8A is an example of the dashboard 810 for an Autism study and management program. In FIG. 8A data is broken down into an overall composite score 811 that is comprise of scores for health/wellness status 813, communication progress or regression 814, social/behavioral progress or regression 815, and academic progress 816. A trending indicator 812 is also shown.

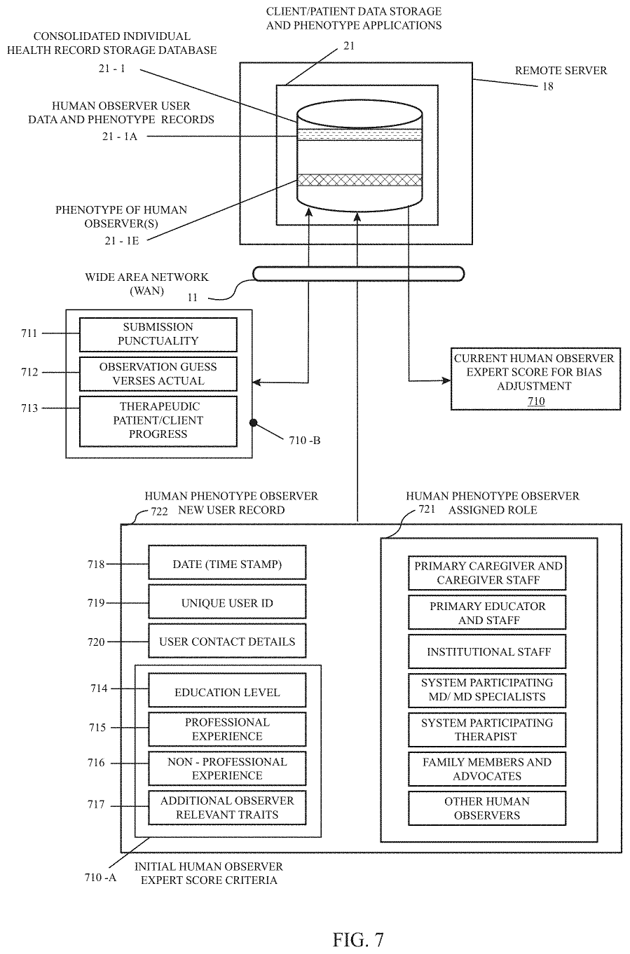

FIG. 9 depicts an autism study/management system menu and observation entry frequency schedule verses action 910 to determine system data reliability.

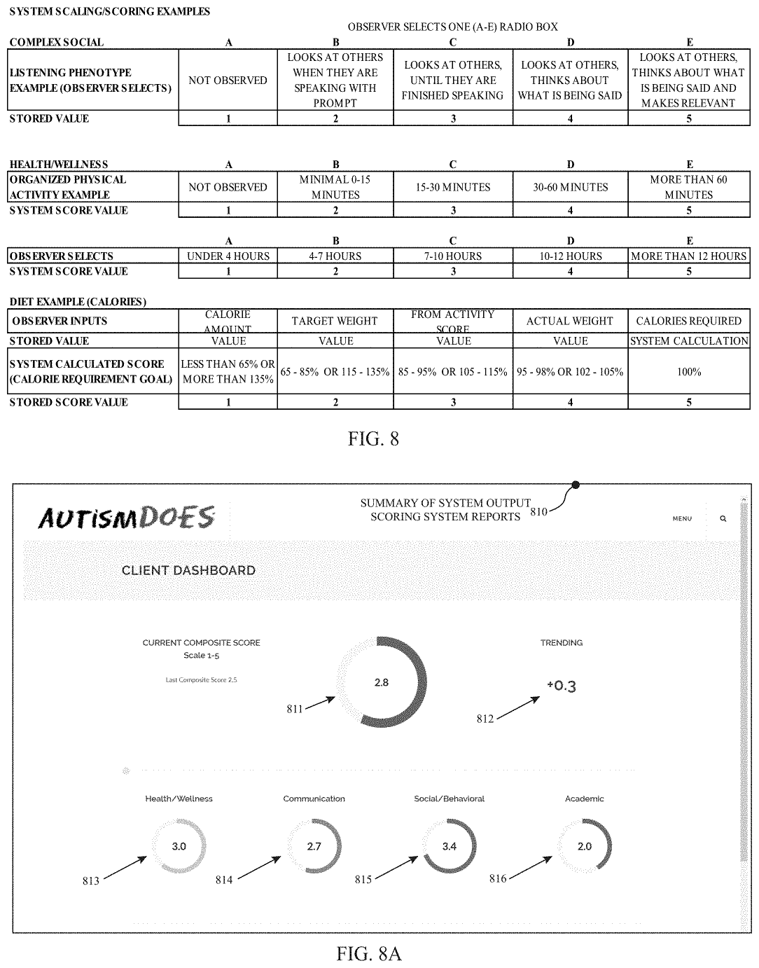

FIG. 10 is the machine learning data input by type and frequency. In FIG. 10. The schedule of inputs and outputs, specific machine learning method, and schedule predictions for feedback to the human phenotype observers.

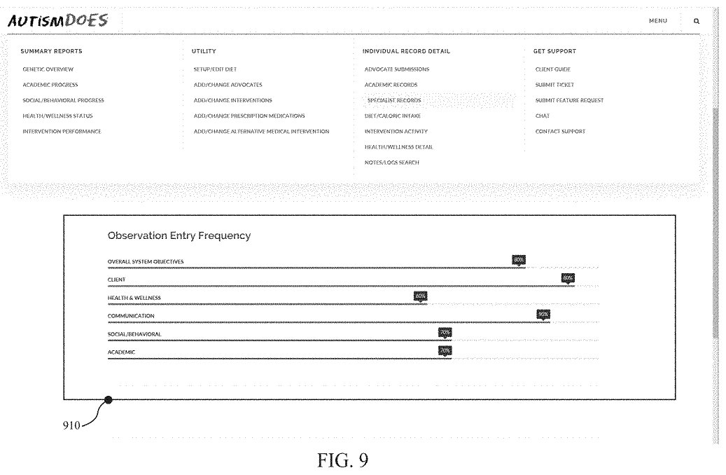

FIG. 11 shows an example of a desired question and possible responses that combine the human phenotype observer and machine learned answer compared to actual for influence of observer expert score 710 and improvement in machine learning score through input of results (back propagation).

### DRAWINGS—REFERENCE NUMERALS

10 electronic health record storage system
10-1 provider(s) remote server
10-2 health record storage database
11 wide area network (WAN)
12 local multiple interfaces/displays
12-1 provider data input and view
12-2 patient/client data portal (view only)
13 patient/client intervention data sources
13-1 therapeutic interventions
13-2 pharmaceutical interventions
13-3 medical procedure interventions
14 patient/client phenotype data sources
14-1 MD/specialist assessment
14-2 known variant genetic testing
14-3 lab test, MRI, and FDA approved diagnostic test results
14-4 medical and family history (pre EI-IR)
15 transcribe/export medical record(s) utility
16 local display
17 non-system medical intervention and phenotype data (historical, current, and future)
18 remote server
19 record input and retrieval
20 patient/client consolidated medical records view
21 patient/client data storage and phenotype system applications
21-1 correlated individual health record storage database
21-1A human phenotype observer data and submission records
21-1B non-human (diagnostic) phenotype record submissions
21-1C intervention records
21-1D genetic test records
21-1E profiles of human observers
21-1F machine learning data storage (interim and results)
23 HIPPA/FERPA secured connection to storage
24 system input interfaces
25 objective human and non-human phenotype submission interface
26 patient/client ID detail
27 user ID role and details
28 health and wellness submission interfaces
29 academic record submission interfaces
30 medical/pharmaceutical intervention submission interfaces
31 therapeutic intervention progress submission interfaces
32 homeopathic/non-FDA intervention submission interfaces
33 planned and actual interventions submission interfaces
34 whole human genome and/or exome upload interfaces
35 observer profile submission interfaces
36 submission record ID
37 machine learning inputs (images, audio, electro biometric sensory devices)
38 subjective human phenotype observer submissions
39 health/wellness attitude/emotional submission interfaces
40 complex social skill communication submission interface
41 complex social skill behavioral submission interface
42 observers note and log submission interfaces
43 observers “guess” submissions interface
44 outside research database(s)
45 application program interfaces
46 data filters (input/output restricted by user role and patient/client ID)
47 system output (reports and analysis)
48 individual submission record detail
49 health/wellness record detail
50 observer submissions
51 medical/pharmaceutical intervention activity
52 academic records
53 therapeutic intervention activity
54 (intentionally left blank)
55 homeopathic non-FDA intervention activity
56 notes/logs search
57 scheduled and actual observation/intervention activity
58 observer “guess” and actual
59 observer expert scores
60 genetic test overview/results
61 individual and clustered data reports
62 health/wellness status
63 therapy/therapist's sessions progress
64 academic/vocational progress
65 complex social skills behavioral progress
66 complex social skills communication progress
67 intervention(s) efficacy determined by direct correlation to progress
68 human observers “guess” versus neural network output
69 view individual patient/client intervention efficacy and progress compared to clustered de-identified system/study participant intervention efficacy and progress records (filtered by phenotype and genotype)
70 related outside research by similar genotype and phenotype
410 observer roles and participation table
710 current human observer expert scores for bias adjustment
710-A initial human observer expert score criteria
710-B human observer score adjustment criteria
711 submission punctuality
712 observer “guess” accuracy
713 therapeutic patient/client progress
714 education level
715 professional experience
716 non-professional experiences
717 additional observer relevant traits
718 date (time stamp)
719 unique user ID
720 user contact details
721 human phenotype observer assigned roles
722 human phenotype observer new user record
810 summary of system output and scoring reports
910 observation entry frequency (system utilization

### Operation

In operation, users of our system can perform various tasks defined by role (please note; 2 and 3 may be combined):
1) System Administrators: Create new patient/client's and add/remove patient/client admin users and assign them to the patient/client. View/edit all users and client contact details.
2) Patient/Client Administrators: Consolidate, manage, and view own patient/client medical records by using the import/transcribe medical record utility 15 to enter current and historical medical records for storage on 21-1 and retrieval 19 for view/print function 20 on a local display 16. Create new human phenotype observer user records 722, add/remove human phenotype observers 721 and view current human phenotype user expert score 710.
3) Primary Caregivers: Enter health/wellness data 28, medical interventions 30, homeopathic interventions 32, scheduled intervention and observation activities 33, defined machine learning input 37, and their own subjective observations 39-43. View all system output records and reports 47 (own or clustered deidentified data via 46).
4) Family Members: Enter health/wellness data 28, and their own subjective observations 38. View own submission records.
5) Participating Medical Doctors/Specialists: Enter health/wellness 28 observations, medical interventions 30, genetic test results 34, notes 42. View own submissions.
6) Primary Educator: Add/remove educator staff users. Enter academic record submissions 29, medical 30 and therapeutic 31 activity (if event occurs under their custodial care), and own subjective human phenotype observations 38. View own submission records, educator staff submission records, own and educator staff expert scores 710, health/wellness status 62, academic/educational progress 64, complex social skills progress 65-66, and observer guess verses neural network output 68.
7) Educator Staff: Enter academic record submissions 29, medical 30 and therapeutic 31 activity (if event occurs under their custodial care), and own subjective human phenotype observations 38. View own submission records, expert score 710, health/wellness status 62, academic/educational progress 64, complex social skills progress 65-66, and own observer guess verses neural network output 68.
8) Vocational/Employers: Enter health/wellness submissions, own subjective human phenotype observer submissions 38. View own submissions, observer “guess” verses neural network output 68, and own expert score 710.
9) Peer Observers: Enter own subjective human phenotype observations 38. View own submission records, observer “guess” verses neural network output 68, and own expert score 710.
10) Institutional Caregivers: Enter health/wellness data 28, medical interventions 30, homeopathic interventions 32, scheduled intervention and observation activities 33, defined machine learning input 37, and their own subjective observations 39-43. View all system output records and reports 47 (own or clustered deidentified data via 46) subject to patient/client administrator.
11) Researchers: Enter related outside research 70 and perform data searches. View own searches, aggregated and deidentified portions of data from observer “guess” versus neural output 68, participant data 62-67, 710-A, 710-B, 710, and perform a plurality of analytics on system data.

### Conclusion, Ramifications, and Scope

Accordingly, the reader will see that the use of this method and system for observing, recording, storing, monitoring, tracking, correlating, and analyzing human phenotype data will improve medical intervention optimization, non-medical intervention optimization, and add utility to human genotype data by:
It makes consolidating medical records and ongoing phenotype records feasible on a patient oriented (individual) basis by providing a utility to do so;
It provides a patient-oriented platform for medical research whereby research participants are the direct beneficiaries from their investment in real time;
It provides an opportunity for greater institution and community involvement in phenotyping;
It economizes healthcare by optimizing invention type and frequency based on effectiveness;
It improves the accuracy of a plurality of human phenotype observers by improving the accuracy of their observations through human and neural network interaction;
It provides, and assigns added value for a plurality of genetic testing through greater phenotype matching, consistency and accuracy;
It acts as a pre-screening tool for potential new and existing intervention therapies by matching genotype and phenotype data;
It improves human medical research by defragmenting phenotype and genotype data whereby increasing the number of participants directly benefits the subjects of the research;
It provides social value for patient/clients, their caregivers, family members, and their advocates by providing a platform to participate together to educate, remediate, potentially resolution of disease, and hope;

Although the description above was conceived for autism spectrum disorder study and management and contains many specificities, these should not be construed as limiting the scope of the embodiments but as merely providing illustrations of some of the presently preferred embodiments. For example, the system could also be used to study other medical disease such as addiction, dementia, and a plurality of other human illnesses and conditions.

Thus, the scope of the embodiments should be determined by the appended claims and their legal equivalents, rather than by the examples given.

1. A method for accurate and continuous collection, management, and validation of human phenotype data comprising:
a. Providing increased human phenotype data accuracy by means for increased collection through the inclusion and aggregation of data from a greater number of observers;
b. Providing consolidated individual phenotype data input, individual and aggregated output data access by means of central storage and single point phenotype data input and output interface access from a plurality of medical and non-medical observation sources;
c. Providing validation of human phenotype data by means of providing feedback to human observers to improve their phenotype observation skill, accuracy and harvest their intuition;

2. A method to compress the medical research discovery timeline comprising:
a. Providing greater human research subject participation by means of real-time information and direct benefit to research participants and stakeholders;
b. Providing for research subject awareness of interventions that are being tested or applied by other research subjects within similar phenotype and genotype by means of comparison of other users within the same disease category;
c. Providing researchers with access to larger phenotype datasets to correlate with genotype data by means of deidentified phenotype and genotype data access;

3. A method of optimizing and identifying intervention efficacy in a plurality of medical and non-medical applications comprising;
a. Providing increased intervention efficacy assessment by means of consistent intervention activity input and phenotype responses to said intervention activity on a consistent timeline;
b. Providing detailed phenotypical responses to all intervention activity by means of a reporting interface that can be shared with healthcare professions increasing the utility of healthcare visits;
